(19) United States
(12) Patent Application Publication
(10) Pub. No.: US 2004/0258702 A1
(43) Pub. Date: Dec. 23, 2004

(54) Vaccine delivery

(76) Inventors: Joan P. Blonder, Lafayette, CO (US); Claire M. Coeshott, Denver, CO (US); Timothy C. Rodell, Aspen, CO (US); Wren H. Schauer, Boulder, CO (US); Gary J. Rosenthal, Lafayette, CO (US)

Correspondence Address: MARSH FISCHMANN & BREYFOGLE LLP, Suite 411, 3151 S. Vaughn Way, Aurora, CO, 80014, US

(21) Appl. No.: 10/828,842

(22) Filed: Apr. 21, 2004

Related U.S. Application Data
(63) Continuation-in-part of application No. 09/888,235, filed on Jun. 22, 2001.
Application No. 09/888,235 is a continuation-in-part of application No. 09/602,654, filed on Jun. 22, 2000, now abandoned.
(60) Provisional application No. 60/278,267, filed on Mar. 23, 2001.

(51) Int. Cl. A61K 39/00; A61K 39/38; A61K 45/00; A61K 47/00

(52) U.S. Cl. 424/184.1

(57) ABSTRACT

We have developed a vaccine delivery system based on the non-ionic block copolymer, Pluronic®F127 (F127), combined with selected immunomodulators. F127-based matrices are characterized by a phenomenon known as reverse thermogelation, whereby the formulation undergoes a phase transition from liquid to gel upon reaching physiological temperatures. Protein antigens (tetanus toxoid (TT), diphtheria toxoid (DT) and anthrax recombinant protective antigen (rPA) were formulated with F127 in combination with CpG motifs or chitosan, as examples of immunomodulators, and were compared to more traditional adjuvants in mice.

IgG antibody responses were significantly enhanced by the F127/CpG and F127/chitosan combinations compared to antigens mixed with CpGs or chitosan alone. In addition, the responses were significantly greater than those elicited by aluminum salts. Furthermore, the functional activity of these antibodies was demonstrated using either in vivo tetanus toxin challenge or an anthrax lethal toxin neutralization assay. These studies suggest that a block-copolymer approach could enhance the delivery of a variety of clinically useful antigens in vaccination schemes.

### CROSS-REFERENCE TO RELATED APPLICATIONS

This application is a continuation-in-part of U.S. patent application Ser. No. 09/888,235 entitled “DELIVERY VEHICLE COMPOSITION AND METHODS FOR DELIVERING ANTIGENS AND OTHER DRUGS” filed June 22, 200, which U.S. patent application Ser. No. 09/888,235 is a continuation-in-part of U.S. patent application Ser. No. 09/602,654 entitled “IMMUNOGEN COMPOSITION AND METHODS FOR USING THE SAME” filed Jun. 22, 2000 and also claims priority from U.S. Provisional Patent Application Ser. No. 60/278,267 entitled “IMMUNOGEN COMPOSITION AND METHODS FOR DELIVERY OF ANTIGEN TO ELICIT MUCOSAL IMMUNE RESPONSE” filed Mar. 23, 2001, and the entire contents of each and all of these referenced Patent Applications are incorporated by reference herein as if set forth herein in full. Moreover, the subject matter disclosed in each of these referenced Patent Applications is useful in combination with the subject matter disclosed herein, and all combinations of any feature or features disclosed in any of these referenced Patent Applications with any feature or features disclosed herein are within the scope of the present invention.

### FIELD OF THE INVENTION

The invention relates to vaccine delivery, including vaccine delivery vehicle compositions, manufacture of such delivery vehicle compositions and treatments involving such delivery vehicle compositions.

1. INTRODUCTION

Although significant progress in vaccine development and administration has been made, alternative approaches that enhance the efficacy and safety of vaccine preparations remain under investigation. Sub-unit vaccines such as recombinant proteins and synthetic peptides are emerging as novel vaccine candidates. However, traditional vaccines, consisting of attenuated pathogens and whole inactivated organisms, contain impurities and bacterial components capable of acting as adjuvants, an activity which these subunit vaccines lack. Therefore the efficacy of highly purified sub-unit vaccines will require addition of potent adjuvants.

Currently, aluminum compounds are the only adjuvants approved for use in human vaccines in the United States [1]. Despite their good safety record, they are relatively weak adjuvants [1] and often require multiple dose regimens to elicit antibody levels associated with protective immunity. Aluminum compounds may therefore not be ideal adjuvants for the induction of protective immune responses to sub-unit vaccines. Although many candidate adjuvants are presently under investigation, they suffer from a number of disadvantages including toxicity in humans and requirements for sophisticated techniques to incorporate antigens.

We have recently reported the immunostimulatory effects on the mucosal immune response of a unique adjuvant system composed of the block co-polymer, Pluronic®F 127 (F127), and the cationic polysaccharide, chitosan [2]. F127 is a non-ionic, hydrophilic polyoxyethylene-polypropylene (POE-POP) block copolymer previously used for its surfactant and protein stabilizing properties [3-5]. F127-based matrices are characterized by a phenomenon known as reverse thermo-gelation whereby they undergo a phase transition from liquid to gel upon reaching physiological temperatures. Therefore formulations of F127 can be administered in liquid form at temperatures less than approximately 10° C., with conversion to semi-solid gels at body temperature, thereby potentially acting as sustained release depots. Furthermore, proteins contained within the pluronic matrix at high concentrations have been shown to retain their native configuration [3].

Chitosan has previously been shown to have both mucosal and systemic adjuvant activity [6-9]. We used a F127/chitosan combination as a delivery vehicle for mucosal vaccine administration and demonstrated that both components contributed to the immunoenhancing effect observed [2]. In the present studies, we demonstrate the utility of the F127/chitosan system as a vaccine delivery vehicle for protein antigens for systemic immunization. In addition, in order to evaluate the potential of F127 to enhance the activity of other adjuvants, we incorporated immunostimulatory DNA preparations containing CpG motifs (CpGs) [10-13] into the formulations and show here that the activity of this adjuvant was dramatically enhanced within the pluronic matrix. Furthermore, these formulations elicit protective antibody responses. Although both chitosan and CpGs are known to have potent adjuvant activity, the combination of either with F127 is unique and results in improved immune responses compared to either adjuvant used alone. To prepare formulations, vaccine antigens and immunomodulators are simply mixed with the vehicle. This straightforward approach may therefore enhance delivery of a variety of clinically useful antigens in vaccination schemes.

2. Materials And Methods

2.1 Antigens

Tetanus toxoid (TT), was obtained from Accurate Chemical & Scientific (Westbury, N.Y.), and contained 961 Lf/ml and 1884 Lf/mg protein nitrogen. Diphtheria toxoid (DT; Accurate) contained 2100 Lf/ml and 1667 Lf/mg protein nitrogen. Recombinant anthrax protective antigen (rPA) was obtained from Dr. Stephen Leppla (NIH), under a license agreement with the NIH, as a lyophilized protein in 5 mM Hepes, pH 7.4. It was reconstituted in water (USP grade; Abbott Laboratories, Chicago, Ill.) at 2 mg/ml before use.

2.2 Preparation of Formulations

Pluronic® F127 (BASF, Washington, N.J.) stock solution was prepared at 30 or 34% (w/w) in ice-cold PBS with complete dissolution achieved by storing overnight (ON) at 4° C. Chitosan (medium molecular weight chitosan; Sigma-Aldrich, St. Louis, Mo.) or Protasan® (Chitosan chloride, UP CL 213; ProNova Biomedical, Oslo, Norway) stock solution was prepared at 3% (w/w) in 1% (v/v) acetic acid in 0.9% (w/v) saline and heated at 37° C. to dissolve. These sources of chitosan had equivalent activity in our formulations. Proprietary preparations of oligodeoxynucleotides containing CpG dinucleotide motifs (CpGs) were obtained from Qiagen (ImmunEasy™; Qiagen Inc., Valencia, Calif.) and were added to formulations or mixed with antigens alone according to the manufacturer's instructions. This proprietary preparation of CpG additionally contains aluminum hydroxide. Unless otherwise noted, the stock solutions were mixed together to prepare formulations containing various combinations of antigen, 0.5% (w/w) chitosan, 20% (v/v) CpGs and 16.25% (w/w) F127.

TT adsorbed to aluminum phosphate (AP; Wyeth Laboratories Inc., Marietta, Pa.) was obtained as a preparation containing 10 Lf/ml. rPA/alum was prepared by adsorption of rPA to Imject® alum (Pierce Endogen, Rockford, Ill.) by standard methods.

To prepare emulsions with Incomplete Freund's Adjuvant (IFA; Sigma-Aldrich), antigens with or without immunomodulators were diluted in PBS and emulsified with IFA at a 1:1 (v/v) ratio.

2.3 Immunization Studies in Mice

Balb/c female mice (Taconic Farms Inc., Germantown, N.Y. or Harlan S.Dak., Indianapolis, Ind.) and ICR (CD-1®) outbred female mice (Harlan), 6 to 8 weeks of age, were used for these studies. Mice were immunized once intra-peritoneally (i.p.) or subcutaneously (s.c) with various formulations as described above.

2.4 ELISA

The serum antibody responses to TT, DT and rPA were measured by ELISA as previously described [2]. Briefly, serum samples were obtained by bleeding from the retro-orbital plexus under inhalation anesthesia and were stored at −20° C. until assay. Wells of 96 well Nunc Maxisorb microtiter plates (Nunc, Gaithersburg, Md.) were coated with either 1 μg/ml TT or rPA or 1 μg/ml DT in PBS. Plates were washed with PBS/0.05% Tween 20 and blocked with 1% bovine serum albumin (BSA; Fisher Scientific, Pittsburgh, Pa.). Samples were serially diluted in PBST (PBS/0.1% BSA/0.05% Tween 20) and added to wells in triplicate. Following incubation, plates were washed and goat anti-mouse IgG-gamma chain specific horseradish peroxidase (HRP)-labeled conjugate (Sigma-Aldrich) was added in PBST. After further incubation, antibody binding was detected with substrate buffer containing TMB (3,3′,5,5′tetramethylbenzidine; Sigma-Aldrich). After the reaction was stopped with 0.5 M H₂SO₄ (Sigma-Aldrich), absorbance was read at 450 nm with an EIA reader (Molecular Devices, Sunnyvale, Calif.). Assays to measure antibody IgG subclasses were performed as described above using IgG1 and IgG2a specific HRP-labeled conjugates (Southern Biotechnology Associates, Birmingham, Ala.). Antibody titer was defined as the reciprocal of the dilution of serum that would yield an optical density of 0.5.

Analysis of differences in titers between groups was performed using the Mann-Whitney Rank Sum Test. A probability (p) of 0.05 or less was accepted as significant.

2.5 ELISPOT assay for Anti-TT Antibody-Secreting Cells (ASC)

Numbers of TT-specific ASC were assessed by ELISPOT assay. Wells of flat-bottomed microtiter plates were coated as described above, blocked with 0.1% BSA/PBS and then washed with PBS before addition of cells. Single cell suspensions from bone marrow and spleen were prepared in Hank's balanced salts solution (BSS; Invitrogen, Carlsbad, Calif.). Bone marrow was obtained from the femurs of immunized or control mice according to the method of Mishell and Shigii [14] and erythrocytes removed with lysing buffer. Cells were washed and resuspended in 5% fetal bovine serum (FBS; Hyclone, Ogden, Utah) in RPMI (Invitrogen) at 5×10⁶ cells/ml. For enumeration of IgG anti-TT ASC, goat anti-mouse IgG (gamma-chain specific) antibody (Kirkegaard and Perry Laboratories (KPL), Gaithersburg, Md.) was added to the cell suspensions at a final dilution of 1:500. Cells were plated at 1.25, 2.5 and 5×10⁵ cells/well in triplicate and plates incubated in a humidified incubator with 5% CO₂ for 3 hr at 37° C. After incubation, plates were washed with 0.01% Tween/PBS and phosphatase-labeled rabbit anti-goat IgG antibody (KPL) was added. Plates were incubated ON at RT and washed before addition of the substrate, BCIP (5-bromo 4chloro 3-indolyl phosphate; Sigma-Aldrich), dissolved at 1 mg/ml in AMP (2-amino-2-methyl-1-propanol; Sigma-Aldrich) buffer, pH 10.25, 0.01% Triton X-100. Plates were developed at RT for 1-2 h and rinsed with distilled water. Spots were counted with the aid of a dissecting microscope at 50× magnification. Results are expressed for individual animals as mean ASC/10⁶ cells.

2.6 Anthrax Toxin Neutralization Assay (TNA)

Serum samples from animals immunized with rPA were tested for their ability to prevent the lethal toxin (PA+lethal factor (LF))-induced mortality of J774A.1 cells (American Type Culture Collection, Mamssas, Va.) [15]. LF was obtained from NIH under an MTA. Aliquots of 100 μl cell suspension (6 to 8×10⁵ cells/ml) in Dulbecco's modified Eagle's medium with 10% FBS (Invitrogen) were plated into flat 96-well cell culture plates (Corning Costar, Acton, Mass.). Serial dilutions of pre- and post-immune serum samples were made in TSTA buffer (50 mM Tris pH 7.6, 142 mM sodium chloride, 0.05% sodium azide, 0.05% Tween 20, 2% BSA). PA and LF at final concentrations of 50 and 40 ng/ml respectively were added to each antiserum dilution. After incubation for 1h, 10 μl of each of the antiserum toxin complex mixtures were added to 100 μl J774A. 1 cell suspension. The plates were incubated for 5h at 37° C. in 5% CO₂ Twenty-five μl of MTT (3-[4,5-dimethyl-thiazol-2-y-]-2,5-diphenyltetrazolium bromide; Sigma-Aldrich) at 5 mg/ml in PBS was then added per well. After 2h incubation, cells were lysed and the reduced purple formazan solubilized by adding 20% (w/v) SDS in 50% dimethylformamide, pH 4.7 [16]. ODs were read at 570 nm on an EIA reader. The lethal toxin-neutralizing antibody titers of individual serum samples, calculated by linear regression analysis, were expressed as the reciprocal of the antibody dilution preventing 50% cell death and these titers were normalized to a control rabbit anti-PA antiserum (from NIH).

Pre and post-immunization serum toxin neutralization titers were compared by the Sign test. Toxin neutralization titers between groups were compared by the use of the Mann Whitney U test. P values less than or equal to 0.05 was considered to indicate a significant difference.

2.7 Tetanus toxin challenge

Lethal challenge with tetanus toxin was performed as described by Anderson et al. [17]. Briefly mice were immunized i.p. on day 0 with 0.5 Lf TT in either PBS or F127/chitosan. Negative controls consisted of mice immunized i.p. with vehicle (F127/chitosan) alone. At 6 weeks, all mice were challenged i.p. with 100×LD₅₀ tetanus toxin (List Biological Labs. Inc., Campbell, Calif.). Mice were monitored for 1 week thereafter and deaths recorded.

### 3. RESULTS

3.1 Duration of the Antibody Response Following S.C. Immunization with TT/AP and TT/F127/Chitosan

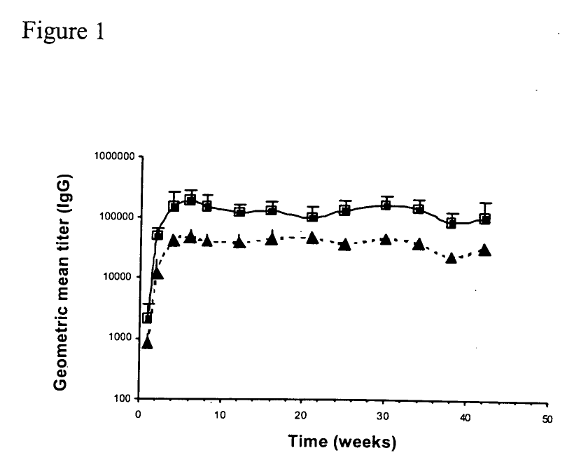

Groups of outbred ICR mice were immunized once s.c. with 1.5 Lf TT formulated either in F127/chitosan or adsorbed to AP. Animals were bled at various times and the IgG anti-TT antibody response was monitored over a ten month period. This dose of TT had previously been selected as optimal in these studies (data not shown). The results of this study (FIG. 1) indicate that TT/F127/chitosan raised a rapid and potent IgG antibody response with antibodies being easily detected at one week. These titers rose to a peak approximately 8 to 12 weeks after injection and were then sustained for at least ten months with titers of approximately 100,000. In contrast, the response to TT/AP was slower to appear and did not attain the levels of TT/F127/chitosan immunized mice. At the peak of the response the titers in AP— immunized mice were only one-third of those of TT/F127/chitosan immunized mice (p<0.05 for all time points).

3.2 The Long-Lived Antibody Response to TT/F127/Chitosan is Maintained by Antibody-Secreting Cells Resident in the Bone Marrow

The durable nature of the antibody response to a single injection of TT/F127/chitosan could be explained either by the persistence of antigen or by long-lived antibody secreting cells (ASC), which reside in the bone marrow [18,19]. We therefore enumerated ASC in the bone marrow and spleens of Balb/c mice that had been immunized one year previously with 1.5 LF TT in F127/chitosan or PBS. The data indicate (Table 1) that ASC were present in the bone marrow one year after immunization with TT/FI27/chitosan whereas none could be detected in the spleens of these mice. In contrast, no ASC were found in either the bone marrow or spleen of mice immunized with TT/PBS. However, early in the response, at one and two weeks post-immunization, ASC were abundant in the spleen and draining lymph nodes but not bone marrow of mice immunized with TT/F127/chitosan (data not shown). By day 28 a distribution of the ASC from the spleen to the bone marrow could be observed (data not shown). Animals receiving vehicle (F127/chitosan) alone had no ASC in bone marrow or spleen at any time points (data not shown).

3.3 Single Dose of TT/F127/Chitosan is MORE Potent than Multiple Injections of TT/AP

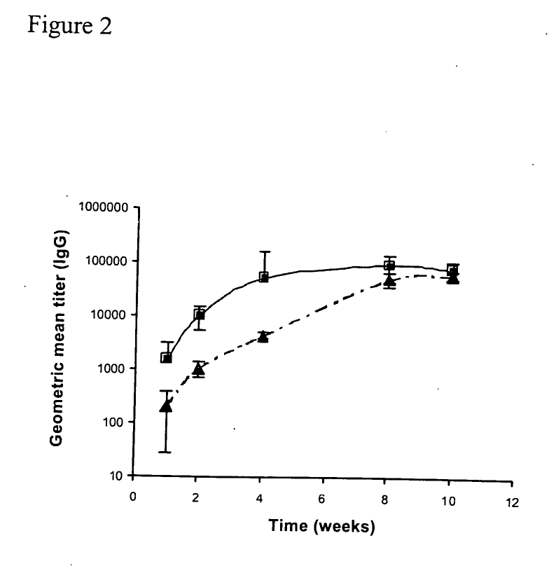

In order to compare our formulation with a standard vaccination regimen, Balb/c mice were immunized s.c. either with a single dose of 1.5 Lf TT/F127/chitosan or with three doses of 1.5 Lf TT/AP given at monthly intervals (total of 4.5 Lf TT given). It is apparent from the data shown in FIG. 2 that the response to TT/AP did not achieve the IgG anti- TT levels of those elicited by TT/F127/chitosan until at least two injections had been administered (p=0.008 at week 2; p=0.012 at week 4; p>0.05 at week 8).

3.4 TT/F127/Chitosan Elicits a Protective Immune Response

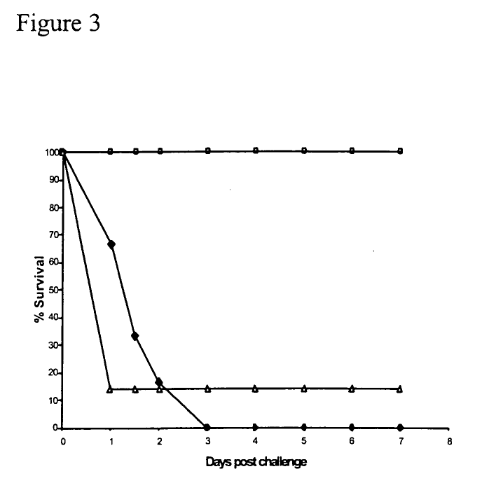

To examine whether these formulations generated a protective immune response, mice were subjected to a lethal challenge with tetanus toxin, performed as described in Anderson et al [17]. Balb/c mice were immunized i.p. with 0.5 LF TT in either PBS or F127/chitosan. In addition, a group of animals received F127/chitosan vehicle alone. At six weeks mice were challenged i.p. with 100×LD₅₀ of tetanus toxin. The results of these studies (FIG. 3) indicate that immunization with TT/F127/chitosan resulted in protective immunity as all mice (8/8) survived. These results were significantly different (p=0.005) from the TT/PBS treated mice, which did not survive the lethal toxin challenge (0/8). As expected, animals immunized with vehicle alone also succumbed to the toxin challenge (0/8 survived).

3.5 TT/F127/Chitosan is Superior to Either Component of the Formulation Alone

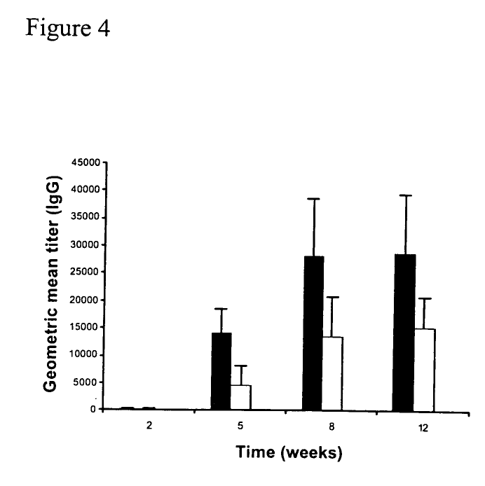

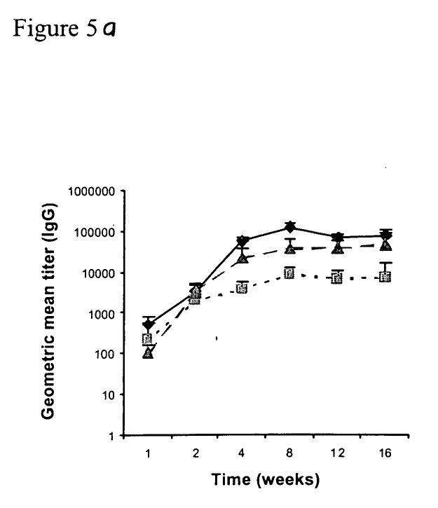

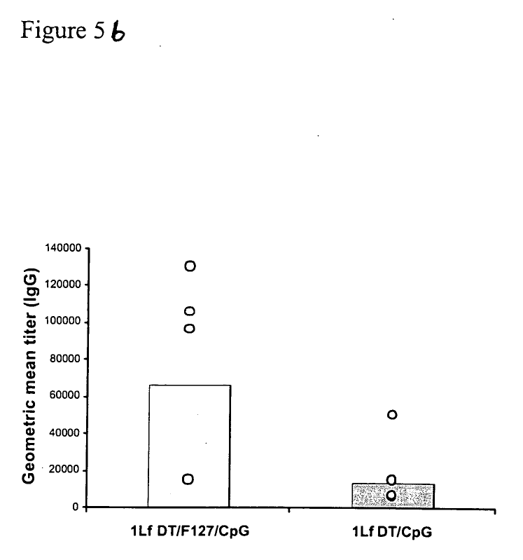

We next compared TT/F 127/chitosan to the same dose of TT given with each component of the formulation mixed with TT alone. In this study, groups of Balb/c mice were given a single s.c. injection of 0.5 Lf TT/F127/chitosan, TT/chitosan or TT/F127. Responses were monitored over a three month period following injection (FIG. 4). TT in the dual component formulation was found to elicit a significantly more potent antibody response 11 than TT/chitosan at 5 weeks after immunization at which time the response to TT/F127/chitosan was approximately 3 times higher than that to TT/chitosan alone (p=0.0206). By week 8, the TT/F127/chitosan response was still twice as high as that to TT/chitosan but this was no longer statistically significant These responses were plateaued at week 8 as they did not increase further by week 12. Also at all times, the responses to TT in both chitosan-containing formulations were significantly greater than that to TT/F127 alone.

3.6 Formulation of CpGs with F127 and Antigen

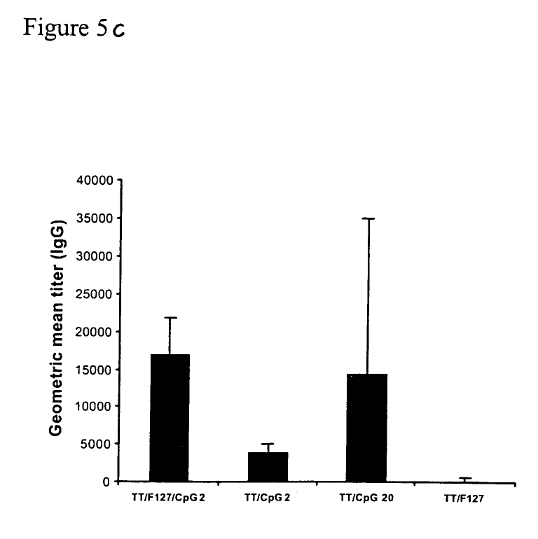

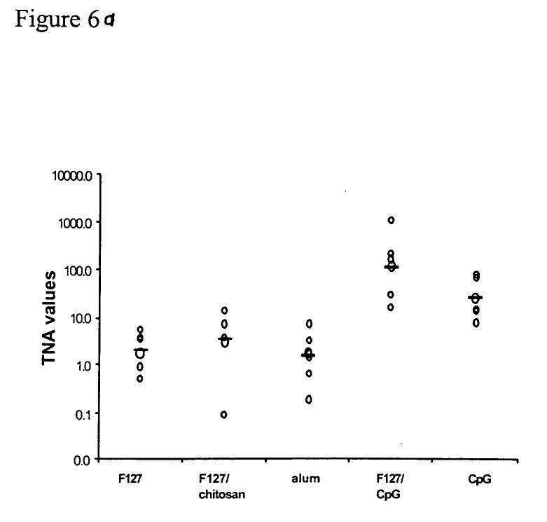

In order to establish whether combinations of F127 with other adjuvants could elicit enhanced responses, groups of Balb/c mice were immunized once s.c. with 0.5 Lf TT either mixed with CpGs or formulated with F127/CpG. In addition, a group of mice was immunized with TT/CpG emulsified in IFA to compare F127 to a classical depot-type adjuvant. Suboptimal doses of the antigens were used in these comparisons to better distinguish between the preparations. Data from a representative experiment (FIG. 5a) indicate that at 4 and 8 weeks, the presence of the pluronic component significantly enhanced the IgG antibody response to TT compared to CpG/antigen alone (p=0.0023 and 0.029 respectively). Furthermore, the response to TT/F127/CpG was significantly higher than that elicited by TT/CpG/IFA (p=0.017 and 0.029 at 4 and 8 weeks respectively).

Similar enhancement was seen when DT was used as the antigen (FIG. 5b). At 4 weeks after a single injection, formulation of DT with F127/CpG elicited a significantly enhanced IgG antibody response compared to that elicited by DT/CpG alone (p<0.05). When the dose of CpGs was reduced in the formulations it was found that, even with a log reduction in the amount of CpG, a better response was still achieved in the presence of F127 (FIG. 5c).

3.7 Formulation of Anthrax rPA in F127 Pluronic

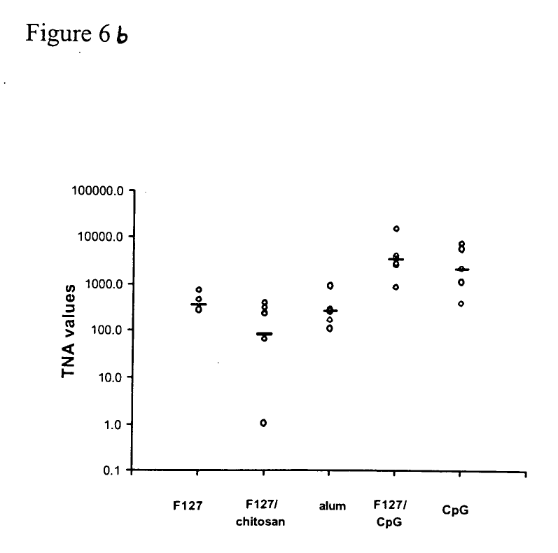

In a preliminary study, we compared the antibody response to a single dose of 25 μg rPA formulated with either F127/chitosan or F127/CpG or adsorbed to alum. In addition a group received the antigen in F127 alone. All animals were boosted 8 months later and the functional nature of the antibody response to rPA was measured by TNA. FIG. 6a shows data from serum samples taken week 8 after the primary injection and demonstrates that formulation of rPA with F127/CpG induced toxin neutralizing titers that were significantly higher than the mix of rPA/CpG alone (p=0.041) as well as rPA/alum (p=0.002), rPAIl 27/chitosan (p=0.001) and rPA/F127 (p=0.002).

At a later time point from same study when samples were taken 2 weeks after the boost (FIG. 6b), all TNA values increased substantially as would be expected. The responses to rPA/F127/CpG and rPA/CpG done were still much higher than all other groups although at this point, there was no significant difference between rPA/F127/CpG and rPA/CpG alone. However, these studies were carried out with a single high dose of rPA (25 μg) and it is likely that this difference could be expanded by the use of limiting doses of antigen and/or adjuvant as illustrated in FIG. 5 with TT as antigen.

Interestingly, after the boost, rPA/F 127 alone could elicit considerable levels of neutralizing antibodies against rPA. Values of approximately 300 were generated, which were similar to those elicited by alum in this study and were higher than the values elicited by the F127/chitosan formulation although these values were not significantly different from each other.

3.8 IgG Subclass Analysis

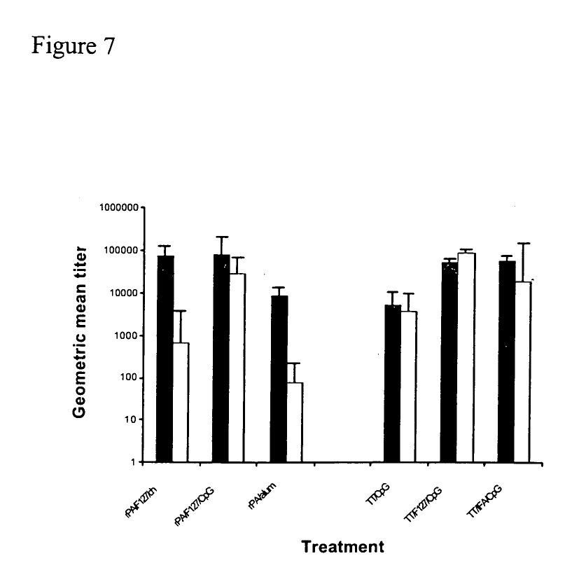

IgG subclass analysis was performed on week 8 sera from mice immunized s.c. with rPA in various formulations. The data indicate that rPA/FI27/chitosan and rPA/alum elicited mainly Th2-type responses with IgG1 being the predominant subclass (FIG. 7). In animals receiving rPA/F127/CpG, the response was dominated by IgG2a indicating that a Th1-type response was elicited as has been previously reported in the literature [10-12]. IgG subclass analysis was also performed on samples from mice immunized s.c. with TT/F127/CpG combinations. These data (FIG. 7) also indicate that CpGs strongly influenced the IgG antibody response, with a significant IgG2a anti-TT response. IgG1 was still easily detectable in all samples, however.

4. DISCUSSION

In a previous study we demonstrated that a novel vaccine delivery system consisting of a sustained release component, Pluronic® F127, combined with a penetration enhancing adjuvant, chitosan, and the antigen, TT, significantly increased the antibody response to intranasally delivered antigen [2]. In this report we establish that this formulation also significantly enhances the antibody response to systemically administered antigens. Furthermore we show that the immunostimulatory activity of another potent adjuvant, CpG, was also significantly enhanced upon formulation in the pluronic matrix.

A single immunization with antigen in F127/chitosan induced an antibody response significantly greater than the immune response to TT/alum in both inbred and outbred mice. Moreover, at least two immunizations with TT/alum were required to induce an anti-TT antibody response comparable to that obtained after a single dose of TT in F127/chitosan. In addition, at early time points, the response to TT/F127/chitosan was significantly higher than that to TT mixed with chitosan in the absence of the pluronic. The duration of the antibody response following a single dose of TT in F127/chitosan, was evaluated over a ten month period and showed minimal decay in antibody levels over time. These results indicate a continual production of anti-TT antibodies as the half-life of IgG is only approximately 23 days [20]. We found that this response was maintained by long-lived antibody-secreting cells, resident in the bone marrow. The generation of these long-lived cells greatly diminishes the degree of regeneration required to maintain persistent antibody levels [21] and thus these cells represent an important first line of defense against re-infection before the memory B cell population is activated to effector stage.

Formulations of TT with F127/chitosan elicited-protective immunity as mice immunized with TT/F127/chitosan survived an otherwise lethal challenge with tetanus toxin six weeks after a single injection, indicating that the antigen was maintained in its native conformational state within the formulation. Taken together with results showing longevity of the immune response after a single immunization, the results suggest that these formulations are capable of eliciting durable, protective antibody responses. Although protection was not monitored at later time points, the lack of diminishment in the antibody levels suggests that protection would be maintained over a long period of time.

The presence of F127 enhanced the immunogenicity of TT administered with chitosan and afforded an early advantage in induction of the IgG antibody response. This enhancement although modest (approximately three-fold) compared to chitosan alone (see FIG. 4), may be due to the ability of F127 to stabilize the protein antigen. We have not investigated if conformation of the protein antigen is maintained in mixtures with chitosan without F127 although McNeela et al. [8] and Seferian and Martinez [9] have reported that combinations of antigen and chitosan can elicit functional antibodies. The improvement of the antibody response at early time points by chitosan in the presence of F127 has previously been seen in intranasal administration of this formulation [2]. However, chitosan was an ineffective adjuvant when used in combination with anthrax rPA (see FIGS. 6a and b). This was probably due to the low resultant pH of this formulation since rPA is a pH sensitive antigen and will unfold at pH less than 6,

The enhanced adjuvant effects of chitosan administered in combination with TT/F127 suggested that F127 might be synergistic with other immunomodulating agents. We therefore also studied the immunogenicity of CpG preparations in combination with TT and F127. The ability of these oligonucleotides to enhance both mucosal and systemic immune responses to a wide variety of antigens is well documented in the literature [10,22-27]. A recent study in mice [22] showed that the combination of other adjuvants with CpGs significantly enhanced the immune response to hepatitis B virus surface antigen (HBsAg). Several adjuvants were tested in combination with CpGs, including alum, IFA, CFA and MPL. The combination of IFA with CpGs resulted in the highest IgG anti-HBsAg antibody response and this response was higher than either component alone. However, the combination of CpGs and alum also induced a synergistic IgG antibody response of similar magnitude to the CpG/IFA combination. In a separate study, using a bovine herpes virus glycoprotein in cattle, combination of CpGs with another oil-in-water based adjuvant, Emulsigen, enhanced the response to antigen compared to CpGs used alone [28]. Combinations of adjuvants with different modes of action can therefore clearly be beneficial in terms of raising optimal immune responses, a point that was recently emphasized (see other papers in this volume). We therefore compared CpGs in combination with F127 and, since the commercial preparation of CpG used here additionally contains alum, we were able to measure the additional effects of F127 delivery on this potent combination. We now show here that the immune responses to TT and DT were significantly increased up to ten-fold (see FIGS. 5a and b) when the antigen was formulated with F127/CpG/alum as compared to antigen/CpG/alum alone. Furthermore the dose of the CpG/alum could also be lowered in the presence of F127 (FIG. 5c). A tenfold reduction of the CpG dose in the presence of F127 induced a higher antibody response to TT than the standard dose of CpG without the F127 matrix. This suggests that other immunomodulators could also be used at reduced doses in the F 127 matrix thereby potentially leading to lower reactogenicity and other side effects.

The mechanism by which F127 augments the activity of antigens and adjuvants contained within its matrix has not been elucidated. The enhanced antibody response may be a consequence of sustained delivery, targeted delivery, improved stability of the protein or immunomodulator contained in the matrix or a combination of all these effects. The ability of F127 to redirect particles to the reticuloendothelial system in general and bone marrow in particular has previously been shown in rabbits [29], a finding that would tend to suggest that targeted delivery has a role to play in the current studies. Some aspects of its use as an adjuvant have previously been documented [30,31]. For example, Spitzer et al. [30] reported that pluronic F127 in combination with a synthetic peptide from Leishmania major could elicit a Th1 response in mice and could elicit durable protection against this organism [30]. However, the effect of peptide alone was not included in this study so the exact role of F127 remains equivocal. Although in our studies addition of the immunomodulators chitosan and CpG enhanced the immunostimulatory capacity of F127 (FIGS. 4 and 5), F127 alone did also elicit a secondary response to rPA (see FIG. 6b) and thus may play a role in the generation and/or recall of memory responses potentially by directing antigens and/or immunomodulators to immunologically relevant tissues. Combinations of poloxamers, including pluronic F127, have recently been shown to enhance aspects of DNA delivery. For example, increased gene expression in mice of plasmid DNA in vivo occurred when the plasmid was formulated in combination of poloxamers that included F127 [32,33] and it was also reported that the mechanism of action centered on the ability to potentiate cellular uptake and to recruit and mature dendritic cells (DCs) [32]. However, these effects were optimal at very low, non-gelling concentrations (0.01% w/v) of poloxamers and thus similar mechanisms may not be operative in the current studies, in which we use much higher concentrations of F127 in combination with CpGs. Other work suggests that F127 can elicit hematopoiesis. For example, a recent study examined the bioavailability of and hematopoietic activity induced by Flt3 ligand (Flt3L) in mice. When delivered in an F127-based matrix, the F127 vehicle alone was found to cause a significant though modest increase in numbers of splenic colony forming units compared to control mice receiving BSS and this activity could not be attributed to endotoxin contamination [34]. Data from a related study indicate that delivery of Flt3L in the F127-based matrix also enhanced numbers of mature DCs in the blood compared to Flt3L delivered in BSS.

However, in both these sets of studies, the formulations additionally contained hydroxypropylmethyl cellulose and therefore this activity cannot be definitively attributed to F127.

Significant enhancement was also seen in the antibody response to TT/F127/CpG versus TT/IFA/CpG over the first three months following single administration. IFA has been shown to cause a depot effect with antigen, thereby potentially allowing sustained release of antigen over an extended period of time. We also evaluated glycerol as an alternative delivery vehicle for TT/CpG because of its known protein stabilizing abilities [35,36] but this caused no enhancement of the anti-TT antibody response compared to TT/CpG alone.

These data therefore suggest that the depot/stabilization effects are not sufficient to explain the enhancement obtained in the presence of F127.

This strongly suggests that the F127 has some inherent properties allowing it to target the immune system. This is further supported by the work of Lemieux and co-workers [32] mentioned above and by our data showing that after a boost, anthrax rPA incorporated in the F127 matrix, without addition of other immunomodulators, elicited a substantial neutralizing antibody response (FIG. 6b), which was equivalent to the secondary response elicited by rPA adsorbed to alum.

The currently available vaccine for anthrax (AVA or BioThrax™), which contains alum as an adjuvant, is considered safe and efficacious [37]. However, it has considerable drawbacks including poor standardization and the requirement for six immunizations over an 18 month period followed by annual boosters to maintain an immune response commensurate with protection [38]. It has also been associated with a considerable number of side effects, ranging from mild local reactions to life-threatening reactions, such as anaphylaxis and shock [39]. Therefore, the Institute of Medicine has recommended that there is an urgent need for the development of a new vaccine.

Several second-generation vaccines based on purified rPA are currently under investigation and/or in clinical trials. Based on a number of animal models, including nonhuman primates, it is widely accepted that the humoral immune response, specifically anti-PA antibodies, plays a significant role in protection against anthrax.

However, the level of anti-PA antibodies necessary to provide protective immunity and the role of cellular immunity are poorly defined. Based on these limitations it seems prudent to design a novel anthrax vaccine capable of inducing both a significant anti-PA antibody response and a cellular immune response. The F127/CpG formulation described here biased the immune response towards a Th1 response but not at the expense of the Th2 response as measured by IgG subclass analysis. Eight weeks after a single injection, the formulation containing rPA with F127/CpG induced toxin neutralizing titers that were significantly higher than all other formulations tested including the mix of rPA/CpG alone. Following a boost rPA/F127/CpG and rPA/CpG induced neutralizing antibody levels that were still significantly higher than levels induced by the other formulations tested although they were no longer significantly different from each other. The ability of the F127/CpG formulations to elicit neutralizing antibody responses and the ability of this formulation, as well as F127 alone, to generate immunological memory after a single immunization, strongly suggests that F127 based formulations have potential for the generation of new and novel anthrax vaccine candidates.

Pluronic F127 belongs to a family of non-ionic block copolymers, known as poloxamers [3,40-46]. Other types of poloxamers have previously been used in various experimental vaccine formulations and have been shown to have potent adjuvant activity, e.g. CRL 1005 [47,48]. However, these polymers are very hydrophobic, having a much larger percentage of polyoxypropylene than F127, and they fail to exhibit reverse gelation characteristics. Furthermore it has been reported that the level of immunomodulatory activity of these polymers decreased when high percentages of POE were used [47]. In contrast, F127 acts as a sustained release vehicle and as a stabilizer for both antigen and adjuvant contained within the matrix. It is therefore distinct both chemically and functionally from these members of the poloxamer family that have previously been evaluated as vaccine delivery candidates.

In summary, our studies demonstrate the synergistic adjuvant effect of chitosan and CpGs co-administered with F127 after systemic administration of various protein antigens. In addition, F127 alone appears to play a role in establishing immunological memory. These promising results have encouraged us to investigate the use of this unique vaccine delivery system with a number of clinically relevant systemic and mucosal antigens, as well as with other adjuvants that could be potentially given at lower doses within the pluronic matrix.

REFERENCES

1 Gupta, R. K., Rost, B. E., Relyveld, E. & Siber, G. R. Adjuvant properties of aluminium and calcium compounds. In Vaccine design, the subunit and adjuvant approach (Eds. Powell, M. F. & Newman, M. J.) Plenum Press, New York, 1995. 229-248.

2 Westerink, M. A. J., Smithson, S. L., Srivastava, N., Blonder, J., Coeshott, C. & Rosenthal, G. J. Projuvant® (Pluronic F127®/chitosan) enhances the immune reponse to intranasally administered tetanus toxoid. Vaccine 2002,20,711-723.

3 Stratton, L. P., Dong, A., Manning, M. C. & Carpenter, J. F. Drug delivery matrix containing native protein precipitates suspended in a poloxamer gel. J Pharm Sci 1997,86(9), 1006-1010.

4 Yao, J., Battell, M. L. & McNeill, J. H. Acute and chronic response to vanadium following two methods of streptozotocin-diabetes induction. Can J Physiol Pharmacol 1997,75(2), 83-90.

5 Wang, P. L. & Johnston, T. P. Enhanced stability of two model proteins in an agitated solution environment using poloxamer 407. Journal of Parenteral Sciences and Technology 1993,47,183-189.

6 Bacon, A., Makin, J., Sizer, P. J. et al. Carbohydrate biopolymers enhance antibody responses to mucosally delivered vaccine antigens [In Process Citation]. Infect Immun 2000, 68(10), 5764-5770.

7 Jabbal-Gill, I., Fisher, A. N., Rappuoli, R., Davis, S. S. & Illum, L. Stimulation of mucosal and systemic antibody responses against Bordetella pertussis filamentous haemagglutinin and recombinant pertussis toxin after nasal administration with chitosan in mice. Vaccine 1998, 16(20), 2039-2046.

8 McNeela, E. A., O'Connor, D., Jabbal-Gill, I. et al. A mucosal vaccine against diphtheria: formulation of cross reacting material (CRM(197)) of diphtheria toxin with chitosan enhances local and systemic antibody and Th2 responses following nasal delivery. Vaccine 2000, 19(9-10), 1188-1198. [MEDLINE record in process].

9 Seferian, P. G. & Martinez, M. L. Immune stimulating activity of two new chitosan containing adjuvant formulations. Vaccine 2000,19(6), 661-668.

10 Chu, RS., McCool, T., Greenspan, N. S., Schreiber, J. R. & Harding, C.V. CpG oligodeoxynucleotides act as adjuvants for pneumococcal polysaccharide-protein conjugate vaccines and enhance antipolysaccharide immunoglobulin G2a (IgG2a) and IgG3 antibodies. Infect Immun 2000, 68(3), 1450-1456.

11 Davis, H. L., Weeratna, R., Waldschmidt, T. J. et al. CpG DNA is a potent enhancer of specific immunity in mice immunized with recombinant hepatitis B surface antigen. J Immunol 1998, 160(2), 870-876.

12 Corral, R. S. & Petray, P. B. CpG DNA as a Th1-promoting adjuvant in immunization against Trypanosoma cruzi. Vaccine 2000,19(2-3), 234-242.

13 Weiner, G. J., Liu, H. M., Wooldridge, J. E., Dahle, C. E. & Krieg, A. M. Immunostimulatory oligodeoxynucleotides containing the CpG motif are effective as immune adjuvants in tumor antigen immunization. Proc Natl Acad Sci USA 1997, 94(20), 10833-10837.

14 In Selected methods in cellular immunology, Vol. xxix (Ed. Shiigi, S. M.) Freeman, 1980. 486.

15 Singh, Y., Chaudhary, V. K. & Leppla, S. H. A deleted variant of Bacillus anthracis protective antigen is non-toxic and blocks anthrax toxin action in vivo. J Biol Chem 1989,264(32), 19103-19107.

16 Hansen, M. B., Nielsen, S. E. & Berg, K. Re-examination and further development of a precise and rapid dye method for measuring cell growth/cell kill. J Immunol Methods 1989, 119(2), 203-210.

17 Anderson, R., Gao, X. M., Papakonstantinopoulou, A., Fairweather, N., Roberts, M. & Dougan, G.

Immunization of mice with DNA encoding fragment C of tetanus toxin. Vaccine 1997, 15(8), 827-829.

18 McHeyzer-Williams, M. G. & Ahmed, R. B cell memory and the long-lived plasma cell. Curr OpinImmunol 1999,11(2), 172-179.

19 Slifka, M. K. & Ahmed, R. Long-lived plasma cells: a mechanism for maintaining persistent antibody production. Curr Opin Immunol 1998, 10(3), 252-258.

20 Basic and Clinical Immunology, Appleton & Lange, Norwalk, Conn., 1991.

21 Slifka, M. K., Antia, R., Whitmire, J. K. & Ahmed, R. Humoral immunity due to long-lived plasma cells. Immunity 1998, 8(3), 363-372.

22 Weeratna, R. D., McCluskie, M. J., Xu, Y. & Davis, H. L. CpG DNA induces stronger immune responses with less toxicity than other adjuvants. Vaccine 2000, 18(17), 1755-1762.

23 McCluskie, M. J. & Davis, H. L. Oral, intrarectal and intranasal immunizations using CpG and non-CpG oligodeoxynucleotides as adjuvants. Vaccine 2000,19(4-5), 413-422.

24 McCluskie, M. J., Weeratna, R. D. & Davis, H. L. The potential of oligodeoxynucleotides as mucosal and parenteral adjuvants. Vaccine 2001, 19(17-19), 2657-2660.

25 McCluskie, M. J., Weeratna, R. D., Payette, P. J. & Davis, H. L. The use of CpG DNA as a mucosal vaccine adjuvant. Curr Opin InvestigDrugs 2001,2(1), 35-39.

26 Krieg, A. M., Love-Homan, L., Yi, A. K. & Harty, J. T. CpG DNA induces sustained IL-12 expression in vivo and resistance to Listeria monocytogenes challenge. J Immunol 1998, 161(5), 2428-2434.

27 Jones, T. R., Obaldia, N., 3rd, Gramzinski, R. A. et al. Synthetic oligodeoxynucleotides containing CpG motifs enhance immunogenicity of a peptide malaria vaccine in Aotus monkeys. Vaccine 1999,17(23-24), 3065-3071.

29 Porter, C. J., Moghimi, S. M., Illum, L. & Davis, S. S. The polyoxyethylene/polyoxypropylene block co-polymer poloxamer-407 selectively redirects intravenously injected microspheres to sinusoidal endothelial cells of rabbit bone marrow. FEBS Lett 1992, 305(1), 62-66.

30 Spitzer, N., Jardim, A., Lippert, D. & Olafson, R. W. Long-term protection of mice against Leishmania major with a synthetic peptide vaccine. Vaccine 1999, 17(11-12), 1298-1300.

31 Reed, C. PhD thesis, University of Strathclyde 1993.

32 Lemieux, P., Guerin, N., Paradis, G. et al. A combination of poloxamers increases gene expression of plasmid DNA in skeletal muscle. Gene Ther 2000,7(11), 986-991.

33 Cho, C. W., Cho, Y. S., Lee, H. K., Yeom, Y. I., Park, S. N. & Yoon, D. Y. Improvement of receptor-mediated gene delivery to HepG2 cells using an amphiphilic gelling agent. Biotechnol Appl Biochem 2000, 32 (Pt 1), 21-26.

34 Robinson, S. N., Chavez, J. M., Pisarev, V. M. et al. Delivery of Flt3 ligand (Flt3L) using a poloxamer-based formulation increases biological activity in mice. Bone Marrow Transplant 2003,31(5), 361-369.

35 Arakawa, T., Prestrelski, S. J., Kenney, W. C. & Carpenter, J. F. Factors affecting short-term and long-term stabilities of proteins. Adv Drug Deliv Rev 2001, 46(1-3), 307-326.

36 Shan, D., Beekman, A., Hartley, C. et al. A G-CSF sustained release formulation using a glycerol oil suspensio: in vivo studies. in 28th International Symposium on Controlled Release of Bioactive Materials, 2001.

37 Joellenbeck, L. M., Zwanziger, L. L., Durch, J. S. & Strom, B. L. (eds.). The anthrax vaccine. Is it safe? Does it work?, National Academy Press, Washington, D.C., U.S.A., 2002, 265.

38 Brachman, P. S., Gold, H., Plotkin, S. A., Fekety, F. R., Werrin, M. & Ingraham, N. R. Field evaluation of a human anthrax vaccine. American Journal of Public Health 1962, 52, 632-645.

39 Swanson-Biearman, B. & Krenzelok, E. P. Delayed life-threatening reaction to anthrax vaccine. J Toxicol Clin Toxicol 2001, 39(1), 81-84.

40 Johnston, T. P., Punjabi, M. A. & Froelich, C. J. Sustained delivery of interleukin-2 from a poloxamer 407 gel matrix following intraperitoneal injection in mice. Pharm Res 1992, 9(3), 425-434.

41 Miyazaki, S., Tobiyama, T., Takada, M. & Attwood, D. Percutaneous absorption of indomethacin from pluronic F127 gels in rats. J Pharm Pharmacol 1995; 47(6), 455-457.

42 Katakam, M. & Banga, A. K. Use of poloxamer polymers to stabilize recombinant human growth hormone against various processing stresses. Pharm Dev Technol 1997,2(2), 143-149.

43 Desai, S. D. & Blanchard, J. Evaluation of pluronic F127-based sustained-release ocular delivery systems for pilocarpine using the albino rabbit eye model. J Pharm Sci 1998,87(10), 1190-1195.

44 Desai, S. D. & Blanchard, J. In vitro evaluation of pluronic F127-based controlled-release ocular delivery systems for pilocarpine. J Pharm Sci 1998,87(2), 226-230.

45 Lee, H. J., Riley, G., Johnson, 0. et al. In vivo characterization of sustained-release formulations of human growth hormone. J Pharmacol Exp Ther 1997,281(3), 1431-1439.

46 Paavola, A., Yliruusi, J., Kajimoto, Y., Kalso, E., Wahlstrom, T. & Rosenberg, P. Controlled release of lidocaine from injectable gels and efficacy in rat sciatic nerve block. Pharm Res 1995, 12(12), 1997-2002.

47 Newman, M. J. Preface. Adv Drug Deliv Rev 1998,32(3), 153-154.

48 Hunter, R. L. & Bennett, B. The adjuvant activity of nonionic block polymer surfactants. Ill. Characterization of selected biologically active surfaces. Scand J Immunol 1986,23(3), 287-300.

TABLE 1
ASC in the bone marrow and spleens of mice one year after
immunization with TT/F127/chitosan or TT/PBS.

| | | |
|---|---|---|
| Source of Cells | TT/F127/chitosan | TT/PBS |

| | | | | | |
|---|---|---|---|---|---|
| Bone marrow | 952 | 384 | 404 | 56 | 120 |
| Spleen | 24 | 8 | 4 | 1 | 1 |

Bone marrow and spleens were obtained from Balb/c mice one year after a single s.c. immunization with 1.5 Lf TT in either F127/chitosan or PBS. ASC were enumerated by ELISPOT assay and data expressed as anti-TT specific ASC/10⁶ cells for individual animals.

FIGURES

FIG. 1

CD-1 mice (n=8) were immunized once s.c. with 1.5 Lf TT/F127/chitosan (squares) or 1.5 Lf TT/AP (triangles). Serum samples were collected at various times and IgG anti-TT antibody levels measured by ELISA. Data are expressed as geometric mean titers of the IgG anti-TT antibody response on a log scale. Error bars represent standard deviations of the mean.

FIG. 2

Balb/c mice were immunized either once s.c. with 1.5 Lf TT/F127/chitosan (n=8) (squares) or three times (0, 4 and 8 weeks) with 1.5 Lf TT/AP (n=4) (triangles) for a total of 4.5 Lf TT. Serum samples were collected at various times and IgG anti-TT antibody levels measured by ELISA. Data are expressed as geometric mean titers of the IgG anti-TT antibody response on a log scale. Error bars represent standard deviations of the nean.

FIG. 3

Balb/c mice (n=8) were immunized i.p. with 0.5 Lf TT in either PBS (diamonds) or F127/chitosan (squares) and were challenged at week 6 with 100×LD₅₀ tetanus toxin.

Negative controls consisted of mice immunized i.p. with vehicle (F127/chitosan) only (open triangles). Survival was monitored for 8 days post challenge and deaths recorded.

FIG. 4

Balb/c mice (n=8) were immunized once s.c. with 0.5 Lf TT in either F127/chitosan, /chitosan or F127. Serum samples were collected at various times and IgG anti-TT antibody levels measured by ELISA.

Data are expressed as geometric mean titers of the IgG anti-TT antibody response. Error bars represent standard errors of the mean. Black bars: TT/F127/chitosan; white bars: TT/chitosan; gray bars: TT/F127.

FIG. 5

Balb/c mice were immunized once s.c. with 0.5 Lf TT (A, C) or 1 Lf DT (B) in various formulations. Serum samples were collected and assayed for IgG antibodies by ELISA. Panel A: IgG anti-TT antibody responses from mice (n=4) immunized with either TT/FI27/CpG (diamonds), TT/IFA/CpG (triangles) or TT/CpG (squares). Data are expressed as geometric mean titers of the IgG anti-TT antibody responses on a log scale. Error bars represent standard deviations of the mean. Panel B: IgG anti-DT antibody responses from mice (n=4) immunized 4 weeks previously. Open circles represent the titers of individual animals; bars represent the geometric mean titers for both groups. Panel C: IgG anti-TT antibody responses from mice (n=8) immunized eight weeks previously either with TT/F127/CpG or TT/CpG at 2% (v/v) CpG or with TT/CpG (20% (v/v)) or with TT/F127 alone. Data are expressed as geometric mean titers of the IgG anti-TT antibody response. Error bars represent standard errors of the mean.

FIG. 6

Balb/c mice (n=6) were immunized with a single s.c. injection of 25 μg of rPA administered in F127, F127/chitosan, F127/CpG, CpG or alum and were boosted s.c. seven months later with the same formulation. Neutralizing antibody titers were measured by TNA in serum samples collected 8 weeks after primary immunization (A) and 2 weeks post boost (B). Open circles represent serum titers from individual mice normalized to a rabbit anti-rPA antiserum control; solid lines represent geometric means of individual normalized TNA values.

FIG. 7

Levels of IgG subclasses were measured by ELISA in serum samples from rpice immunized as described in FIGS. 5A (TT) and 6 (rPA). Data are expressed as geometric mean titers of the IgG anti-TT antibody responses on a log scale. Error bars represent standard deviations of the mean. Black bars: IgG1; white bars: IgG2a.

What is claimed is:

1. An immunogen composition for stimulation of an immune response when administered to a host, the immunogen composition comprising:
an antigen, a biocompatible polymer and a liquid vehicle;
wherein, the polymer interacts with the liquid vehicle to impart reverse thermal viscosity behavior to the composition, so that the viscosity of the composition increases when the temperature of the composition increases over at least some temperature range; and
wherein, the composition further comprises an additive enhancing the immune response when the composition is administered to the host, the additive being selected from the group consisting of a penetration enhancer, an adjuvant and combinations thereof.

2. The immunogen composition of claim 1, wherein the temperature range is below 40° C.

3. The immunogen composition of claim 2 wherein the temperature range is from 1° C. to 37° C.

4. The immunogen composition of claim 2, wherein the composition is in the form of a flowable medium at least when the composition is at a first temperature in the temperature range and the composition is in a gel form at least when the composition is at a second temperature in the temperature range, the second temperature being higher than the first temperature.

5. The immunogen composition of claim 4, wherein the first temperature is in a range of from 1° C. to 20° C.

6. The immunogen composition of claim 3, wherein the first temperature is in a range of from 1° C. to 20° C. and the second temperature is in a range of from 25° C. to 37° C.

7. The immunogen composition of claim 4, wherein the polymer is substantially all dissolved in the liquid vehicle when the immunogen composition is at the first temperature, and at least a portion of the polymer comes out of solution in the liquid vehicle when the temperature of the composition is raised from the first temperature to the second temperature.

8. The immunogen composition of claim 1, wherein the polymer is a polyoxyalkylene block copolymer.

9. The immunogen composition of claim 8, wherein the polyoxyalkylene block copolymer comprises at least one block of a first polyoxyalkylene and at least one of second polyoxyalkylene.

10. The immunogen composition of claim 9 wherein the first polyoxyalkylene is polyoxyethylene and the second polyoxyalkylene is polyoxypropylene.

11. The immunogen composition of claim 10, wherein the polyoxyalkylene block copolymer has the formula:
HO(C₂H₄O)_{b}(C₃H₆O)ₐ(C₂H₄O)_{b}H
wherein a and each b are independently selected integers.

12. The immunogen composition of claim 11, wherein the (C₂H₄O)_{b} blocks together comprise at least 70 weight percent of the polyoxyalkylene block copolymer.

13. The immunogen composition of claim 1 wherein a is between 15 and 80 and each b is independently between 50 and 150.

14. The immunogen composition of claim 10, wherein the polyoxyalkylene block copolymer has the formula: 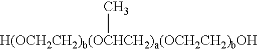
wherein a is 20 to 80 and each b is independently 15 to 60.

15. The immunogen composition of claim 1, wherein the antigen is derived from at least one of bacteria, protozoa, fungus, hookworm, virus and combinations thereof.

16. The immunogen composition of claim 1, wherein the antigen comprises at least one of tetanus toxoid, diphtheria toxoid, a non-pathogenic mutant of tetanus toxoid, a non-pathogenic mutant of diphtheria toxoid and combinations thereof.

17. The immunogen composition of claim 1, wherein the antigen comprises at least one antigen from Bordatella pertussis.

18. The immunogen composition of claim 1, wherein the antigen comprises at least one antigen from influenza virus.

19. The immunogen composition of claim 1, wherein the antigen comprises at least one antigen from M. tuberculosis.

20. The immunogen composition of claim 1, wherein the antigen is derived from at least one causative agent of childhood illness.

21. The immunogen composition of claim 1, wherein the antigen comprises at least one of rotavirus and at least one antigen derived from rotavirus.

22. The immunogen composition of claim 1, wherein the antigen comprises at least one of a polysaccharide, a peptide mimetic of a polysaccharide, or antigen from Neisseria meningitiditis and an antigen from Streptococcus pneumoniae.

23. The immunogen composition of claim 1, wherein the antigen comprises Epstein-Barr virus or at least one antigen derived from Epstein-Barr virus.

24. The immunogen composition of claim 1, wherein the antigen comprises Hepatitis C virus or at least one antigen derived from Hepatitis C virus.

25. The immunogen composition of claim 1, wherein the antigen comprises HIV or at least one antigen derived from HIV

26. The immunogen composition of claim 1, wherein the antigen comprises at least one molecule involved in a mammalian reproductive cycle.

27. The immunogen composition of claim 1, wherein the antigen comprises HCG.

28. The immunogen composition of claim 1, wherein the antigen comprises at least one tumor-specific antigen.

29. The immunogen composition of claim 1, wherein the antigen comprises at least one antigen from a blood-borne pathogen.

30. The immunogen composition of claim 1, wherein the composition contains at least two antigens.

31. The immunogen composition of claim 1, wherein the antigen comprises a first component selected from the group consisting of tetanus toxoid, a nonpathogenic mutant of tetanus toxoid and combinations thereof; and
the antigen comprises a second component selected from the group consisting of diphtheria toxoid, a nonpathogenic mutant of diptheria toxoid and combinations thereof.

32. The immunogen composition of claim 1, wherein the adjuvant comprises products of microorganisms, such as bacteria or yeast, that can enhance uptake and presentation of antigens by antigen presenting cells.

33. The immunogen composition of claim 1, wherein the adjuvant comprises dimethyl dioctadecyl ammonium bromide (DDA).

34. The immunogen composition of claim 1, wherein the adjuvant comprises a CPG motif.

35. The immunogen composition of claim 1, wherein the adjuvant comprises a cytokine.

36. The immunogen composition of claim 1, wherein the adjuvant comprises chitosan material.

37. The immunogen composition of claim 36, wherein the adjuvant comprises N,O-carboxymethyl chitosan.

38. The immunogen composition of claim 1, wherein the liquid vehicle comprises from 60 weight percent to 85 weight percent of the composition, the antigen comprises from 0.0001 weight percent to 5 weight percent of the composition, the polymer comprises from 5 weight percent to 33 weight percent of the composition and the additive comprises from 0.1 weight percent to 1.0 weight percent of the composition.

39. The immunogen composition of claim 1, wherein the composition is in the form of disperse droplets in a mist.

40. The immunogen composition of claim 39, wherein a mist is produced by a nebulizer.

41. The immunogen composition of claim 1, wherein the composition is contained within a nebulizer actuatable to produce a mist comprising dispersed droplets of the composition.

42. The immunogen composition of claim 40, wherein the nebulizer is a nasal nebulizer.

43. The immunogen composition of claim 1, wherein the composition is contained within an injection device that is actuatable to administer the composition to the host by injection.

44. A method of packaging and storing the immunogen composition of claim 5, comprising placing the composition in a container when the composition is in the form of a flowable medium and, after the placing, raising the temperature of the composition in the container to convert the composition to the gel form for storage, wherein the gel form in the container can be converted back to the form of a flowable medium for administration to the host by lowering the temperature of the composition in the container.

45. A delivery vehicle composition comprising:
a drug in an amount effective to produce a desired biological response in a host;
a reverse-thermal gelation biocompatible polymer;
a liquid vehicle in which the polymer is at least partially soluble at some temperature;
an additive selected from the group consisting of a penetration enhancer, an adjuvant and combinations thereof;
wherein proportions of the liquid vehicle and the polymer are such that the composition exhibits reverse thermal viscosity behavior in that the viscosity of the composition increases with increasing temperature over at least some temperature range.

46. The delivery vehicle composition of claim 45, wherein the polymer is a block copolymer.

47. The delivery vehicle composition of claim 45 wherein the block copolymer comprises at least one block of a polyoxyalkylene.

48. The delivery vehicle composition of claim 47, wherein the polyoxyalkylene is a polyoxypropylene.

49. The delivery vehicle composition of claim 47, wherein the polyoyxyalkylene is a polyoxyethylene.

50. The delivery vehicle composition of claim 45, wherein the polymer is a polyoxyalkylene block copolymer.

51. The delivery vehicle composition of claim 50, wherein the polyoxyalkylene block copolymer comprises at least one block of a first polyoxyalkylene and at least one block of a second polyoxyalkylene.

52. The delivery vehicle composition of claim 51, wherein the first polyoxyalkylene is a polyoxyethylene and the second polyoxyalkylene is a polyoxypropylene.

53. The delivery vehicle composition of claim 52, wherein the polyoxyethylene comprise at least 70 weight percent of the polymer.

54. The delivery vehicle composition of claim 52, wherein the polyoxypropylene has the formula (C₃H₆O)_{b}, where b is an integer.

55. The delivery vehicle composition of claim 52, wherein the polyoxypropylene has the formula 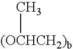
where b is an integer.

56. The delivery vehicle composition of claim 45, wherein the temperature range is within a range of from 1° C. to 37° C.

57. The delivery vehicle composition of claim 45, wherein the composition is in the form of a flowable medium at least at a first temperature and is in the form of a gel at least at a second temperature that is higher than the first temperature.

58. The delivery vehicle composition of claim 57, wherein the second temperature is 37° C. or lower.

59. The delivery vehicle composition of claim 45, wherein the additive comprises from 0.01% by weight to 10% by weight of the composition.

60. The delivery vehicle composition of claim 45, wherein the drug comprises an antigen.

61. The delivery vehicle composition of claim 60, wherein the additive comprises an adjuvant for the antigen, the adjuvant being selected from the group consisting of chitosan material, dimethyl dioctadecyl ammonium bromide (DDA), a CPG motif and a cytokine.

62. The delivery vehicle composition of claim 45, wherein the additive comprises a penetration enhancer selected from the group consisting of chitosan material, poly-L-arginines, fatty acids, salts of fusidic acid, polyoxyethylenesorbitan, sodium lauryl sulfate, polyoxyethylene-9-lauryl ether, citric acid, salicylates, caprylic glycerides, capric glycerides, sodium caprylate, sodium caprate, sodium laurate, sodium glycyrrhetinate, dipotassium glycyrrhizinate, glycyrrhetinic acid hydrogen succinate, disodium salt, acylcamitines, phospholipids, a bacterially-derived product, lysophosphatidylcholine, a CpG motif, a detoxified mutant of CT, a detoxified mutant of ET and an outer membrane protein of Neisseria meningitidis serogroup b.

63. The delivery vehicle composition of claim 45, wherein the additive comprises chitosan material.

64. The delivery vehicle composition of claim 63, wherein the chitosan material comprises at least one of chitosan and a chitosan derivative.

65. The delivery vehicle composition of claim 63, wherein the chitosan material comprises N,O-carboxymethyl chitosan.

66. The delivery vehicle composition of claim 45, wherein the composition is a disperse droplet phase in a mist.

67. The delivery vehicle composition of claim 66, wherein the mist is produced by a nebulizer.

68. The delivery vehicle composition of claim 45, wherein the composition is contained within a nebulizer that is actuatable to produce a mist comprising droplets of the composition.

69. The delivery vehicle composition of claim 68, wherein the nebulizer is a nasal nebulizer.

70. The delivery vehicle composition of claim 45, wherein the composition is contained within an injection device that is actuatable to administer the composition to the host by injection.

71. A method of packaging and storing the delivery vehicle composition of claim 45, comprising placing the composition in a container when the composition is in the form of a flowable medium and then raising the temperature of the composition to convert the composition to a gel form for storage, wherein the gel form in the container can be converted back to the form at a flowable medium for administration to the host by lowering the temperature of the composition in the container.

72. A method for delivery of a drug to a host, the method comprising:
administering a delivery vehicle composition to the host;
the delivery vehicle composition comprising a drug, a reverse thermal gelation biocompatible polymer, a liquid vehicle in which the polymer is at least partially soluble at some temperature, and an additive selected from the group consisting of a penetration enhancer, an adjuvant and combinations thereof;
wherein proportions of the liquid vehicle and the polymer are such that the composition exhibits reverse thermal viscosity behavior in that the viscosity of the composition increases with increasing temperature over at least some temperature range.

73. The method of claim 72, wherein prior to the administering the delivery vehicle composition is at a temperature that is lower than the physiologic temperature of the host;
after the administering the delivery vehicle composition is warmed by the host so that the temperature of the composition increases; and
the delivery vehicle composition is in the form of a flowable medium immediately prior to the administering and the viscosity of the delivery vehicle composition increases after the administering when the temperature of the delivery vehicle composition increases.

74. The method of claim 72, wherein the delivery vehicle composition is in the form of a flowable medium immediately prior to the administering and converts to a gel form after the administering.

75. The method of claim 74, wherein said step of administering the drug delivery composition to the host comprises placing the composition into an injection device and administering the composition to the host by injection.

76. The method of claim 74, wherein at least a portion of the drug delivery composition in the gel form adheres to a mucosal surface, thereby retaining the drug and the additive in the vicinity of the mucosal surface for delivery of the drug across the mucosal surface.

77. The method of claim 76, wherein the mucosal surface is selected from the group consisting of rectal, vaginal, ocular, oral, nasal, intestinal, pulmonary or aural mucosal surfaces.

78. The method of claim 72, wherein the delivery vehicle composition is in the form of dispersed droplets in a mist during the administering.

79. The method of claim 78, wherein the mist is introduced into the nasal cavity of the host during the administering.

80. The method of claim 78, wherein the administering comprises nebulizing the composition to form the mist.

81. The method of claim 72, wherein the drug comprises an antigen to stimulate an immune response in the host.

82. The method of claim 81, wherein after the administering the composition is contacted with a mucosal surface within the host; and the antigen stimulates a mucosal immune response by the host.

83. The method of claim 82, wherein the antigen further stimulates a systemic immune response by the host.

84. The method of claim 83, wherein the administering comprises administering the composition into the nasal cavity of the host and the mucosal surface contacted by the composition is in the nasal cavity.

85. The method of claim 82, wherein the composition is in the form of a flowable medium immediately prior to the administering and converts to a gel form after the administering, so that at least of portion of the composition in the gel form adheres to the musosal surface.

86. The method of claim 81, wherein the additive comprises an adjuvant for the antigen.

87. The method of claim 86, wherein the additive comprises a penetration enhancer.

88. The method of claim 87, wherein the adjuvant and the penetration enhancer are the same material.

89. The method of claim 86, wherein the adjuvant comprises products of microorganisms, such as bacteria or yeast, that can enhance uptake and presentation of antigens by antigen presenting cells.

90. The method of claim 81, wherein the additive comprises an adjuvant selected from the group consisting of dimethyl dioctadecyl ammonium bromide (DDA), a CpG motif, a cytokine, chitosan material and combinations thereof.

91. The method of claim 81, wherein the additive comprises chitosan material.

92. The method of claim 91, wherein the chitosan material is selected from the group consisting of chitosan and a chitosan derivative.

93. The method of claim 91, wherein the chitosan material comprises N,O-carboxymethyl chitosan.

94. The method of claim 87, wherein the additive comprises a penetration enhancer selected from the group consisting of chitosan material, poly-L-arginines, fatty acids, salts of fusidic acid, polyoxyethylenesorbitan, sodium lauryl sulfate, polyoxyethylene-9-lauryl ether, citric acid, salicylates, caprylic glycerides, capric glycerides, sodium caprylate, sodium caprate, sodium laurate, sodium glycyrrhetinate, dipotassium glycyrrhizinate, glycyrrhetinic acid hydrogen succinate, disodium salt, acylcarnitines, phospholipids, a bacterially-derived product, lysophosphatidylcholine, a CpG motif, a detoxified mutant of CT, a detoxified mutant of ET and an outer membrane protein of Neisseria meningitidis serogroup b.

95. The method of claim 81, wherein the antigen is derived from at least one of bacteria, protozoa, fungus, hookworm, virus and combinations thereof.

96. The method of claim 81, wherein the antigen comprises at least one of tetanus toxoid, diphtheria toxoid, a non-pathogenic mutant of tetanus toxoid, a non-pathogenic mutant of diphtheria toxoid and combinations thereof.

97. The method of claim 81, wherein the antigen comprises at least one antigen from Bordatella pertussis.

98. The method of claim 81, wherein the antigen comprises at least one antigen from influenza virus.

99. The method of claim 81, wherein the antigen comprises at least one antigen from M. tuberculosis.

100. The method of claim 81, wherein the antigen is derived from at least one causative agent of childhood illness.

101. The method of claim 81, wherein the antigen comprises at least one of rotavirus and at least one antigen derived from rotavirus.

102. The method of claim 81, wherein the antigen comprises at least one of a polysaccharide, a peptide mimetic of a polysaccharide, or antigen from Neisseria meningitiditis and an antigen from Streptococcus pneumoniae.

103. The method of claim 81, wherein the antigen comprises Epstein-Barr virus or at least one antigen from Epstein-Barr virus.

104. The method of claim 81, wherein the antigen comprises Hepatitis C virus or at least one antigen from Hepatitis C.

105. The method of claim 81, wherein the antigen comprises HIV or at least one antigen from HIV.

106. The method of claim 81, wherein the antigen comprises at least one molecule involved in a mammalian reproductive cycle.

107. The method of claim 81, wherein the antigen comprises HCG.

108. The method of claim 81, wherein the antigen comprises at least one tumor-specific antigen.

109. The method of claim 81, wherein the antigen comprises at least one antigen from a blood-borne pathogen.

110. The method of claim 81, wherein the drug contains at least two antigens.

111. The method of claim 81, wherein the antigen comprises a first component selected from the group consisting of tetanus toxoid, a nonpathogenic mutant of tetanus toxoid and combinations thereof; and
the antigen comprises a second component selected from the group consisting of diphtheria toxoid, a nonpathogenic mutant of diphtheria toxoid and combinations thereof.

112. The method of claim 81, wherein the immune response is a booster to a previous primary immunization of the host.

113. The method of claim 112, wherein at least a portion of the delivery vehicle composition adheres to a mucosal surface within the host, thereby retaining the drug and the additive in the vicinity of the mucosal surface for delivery of the drug across the mucosal surface.

114. The method of claim 112, wherein the magnitude of the immune response is the same or greater than a comparison immune response generated by administering in the same manner as the delivery vehicle composition a comparison composition that is the same as the delivery vehicle composition except being in the absence of one or both of the polymer and the additive.

115. The method of claim 114, wherein the comparison composition is in the absence of both the polymer and the additive.

116. The method of claim 72, wherein the additive comprises chitosan material.

117. The method of claim 72, wherein the temperature range is below 40° C.

118. The method of claim 72, wherein the composition is in the form of a flowable medium at a first temperature in a range of from 1° C. to 20° C. and is in a gel form at a second temperature that is higher than the first temperature.

119. The method of claim 118, wherein the second temperature is 37° C. or lower.

120. The method of claim 72, wherein the polymer is a block copolymer.

121. The method of claim 120, wherein the block copolymer comprises at least one block of a polyoxyalkylene.

122. The method of claim 121, wherein the polyoxyalkylene is a polyoxypropylene.

123. The method of claim 121, wherein the polyoxyalkylene is a polyoxyethylene.

124. The method of claim 120, wherein the polymer is a polyoxyalkylene block copolymer.

125. The method of claim 124, wherein the polyoxyalkylene block copolymer comprises at least one block of a first polyoxyalkylene and at least one block of a second polyoxyalkylene.

126. The method of claim 124, wherein the first polyoxyalkylene is a polyoxyethylene and the second polyoxyalkylene is a polyoxypropylene.

127. A method for delivery of an antigen to a host to stimulate an immune response in the host, the method comprising:
introducing an immunogen composition into a host and the immunogen comprising an antigen, a reverse thermal gelation biocompatible polymer, a liquid vehicle in which the polymer is at least partially soluble at some temperature, and an additive selected from the group consisting of a penetration enhancer, an adjuvant and combinations thereof for enhancing the immune response;
wherein, immediately prior to the introducing the composition is in the form of a flowable medium at a first temperature below the physiologic temperature of the host, and after the introducing the composition warms within the host to at least a second temperature at which the composition is in the form of a gel.

128. The method of claim 127, wherein said steps of introducing the immunogen composition into the host comprises placing the composition into an injection device and administering the composition to the host by injection.

129. The method of claim 127 wherein the method comprises contacting the immunogen composition with a mucosal surface of a host and during the contacting at least a portion of the gel adheres to the mucosal surface whereby at least a portion of the antigen and the additive are retained in the vicinity of the mucosal surface for delivery of the antigen across the mucosal surface.

130. The method of claim 129, wherein the mucosal surface is selected from the group consisting of rectal, vaginal, ocular, oral, nasal, intestinal, pulmonary or aural mucosal surfaces.

131. The method of claim 127, wherein the first temperature is less than 37° C.

132. The method of claim 127, wherein the second temperature is 37° C. or less.

133. The method of claim 129, wherein the drug delivery composition is in the form of dispersed droplets in a mist during the administering.

134. The method of claim 133, wherein the mist is introduced into the nasal cavity of the host during the introducing.

135. The method of claim 134, wherein the introducing comprises nebulizing the composition to form the mist.

136. The method of claim 129, wherein the composition stimulates a mucosal immune response in the host.

137. The method of claim 136, wherein the composition also stimulates a systemic immune response in the host.

138. The method of claim 127, wherein the additive comprises an adjuvant for the antigen.

139. The method of claim 127, wherein the additive comprises a penetration enhancer.

140. The method of claim 127 wherein the adjuvant comprises products of microorganisms, such as bacteria or yeast, that can enhance uptake and presentation of antigens by antigen presenting cells.

141. The method of claim 127, wherein the adjuvant comprises dimethyl dioctadecyl ammonium bromide (DDA).

142. The method of claim 127, wherein the adjuvant comprises a CPG motif.

143. The method of claim 127 wherein the adjuvant comprises a cytokine.

144. The method of claim 127, wherein the adjuvant comprises chitosan material.

145. The method of claim 144, wherein the chitosan material is selected from the group consisting of chitosan and a chitosan derivative.

146. The method of claim 144, wherein the chitosan material comprises N,O carboxymethyl chitosan.

147. The method of claim 127 wherein the additive comprises a penetration enhancer selected from the group consisting of chitosan material, poly-L-arginines, fatty acids, salts of fusidic acid, polyoxyethylenesorbitan, sodium lauryl sulfate, polyoxyethylene-9-lauryl ether, citric acid, salicylates, caprylic glycerides, capric glycerides, sodium caprylate, sodium caprate, sodium laurate, sodium glycyrrhetinate, dipotassium glycyrrhizinate, glycyrrhetinic acid hydrogen succinate, disodium salt, acylcamitines, phospholipids a bacterially-derived product, lysophosphatidylcholine, a CpG motif, a detoxified mutant of CT, a detoxified mutant of ET and an outer membrane protein of Neisseria meningitidis serogroup b.

148. The method of claim 127, wherein the adjuvant and the penetration enhancer are the same material.

149. The method of claim 127, wherein the antigen is derived from at least one of bacteria, protozoa, fungus, hookworm, virus and combinations thereof.

150. The method of claim 127, wherein the antigen comprises at least one of tetanus toxoid, diphtheria toxoid, a non-pathogenic mutant of tetanus toxoid, a non-pathogenic mutant of diphtheria toxoid and combinations thereof.

151. The method of claim 127, wherein the antigen comprises at least one antigen from Bordatella pertussis.

152. The method of claim 127, wherein the antigen comprises at least one antigen from influenza virus.

153. The method of claim 127, wherein the antigen comprises at least one antigen from M. tuberculosis.

154. The method of claim 127, wherein the antigen is derived from at least one causative agent of childhood illness.

155. The method of claim 127, wherein the antigen comprises at least one of rotavirus and at least one antigen derived from rotavirus.

156. The method of claim 127, wherein the antigen comprises at least one of a polysaccharide, a peptide mimetic of a polysaccharide, or antigen from Neisseria meningitiditis and an antigen from Streptococcus pneumoniae.

157. The method of claim 127, wherein the antigen comprises Epstein-Barr virus or at least one antigen derived from Epstein-Barr virus.

158. The method of claim 127, wherein the antigen comprises Hepatitis C virus or at least one antigen derived from Hepatitis C virus.

159. The method of claim 127, wherein the antigen comprises HIV or at least one antigen derived from HIV.

160. The method of claim 127, wherein the antigen comprises at least one molecule involved in a mammalian reproductive cycle.

161. The method of claim 127, wherein the antigen comprises HCG.

162. The method of claim 127, wherein the antigen comprises at least one tumor-specific antigen.

163. The method of claim 127, wherein the antigen comprises at least one antigen from a blood-borne pathogen.

164. The method of claim 127, wherein the immunogen contains at least two antigens.

165. The method of claim 127, wherein the antigen comprises a first component selected from the group consisting of tetanus toxoid, a nonpathogenic mutant of tetanus toxoid and combinations thereof;
the antigen comprises a second component selected from the group consisting of diphtheria toxoid, a nonpathogenic mutant of diphtheria toxoid and combinations thereof.

166. The method of claim 127, wherein the antigen comprises a first component selected from the group consisting of tetanus toxoid, a nonpathogenic mutant of tetanus toxoid and combinations thereof;
the antigen comprises a second component selected from the group consisting of diphtheria toxoid, a nonpathogenic mutant of diphtheria toxoid and combinations thereof; and the adjuvant comprises chitosan material.

167. The method of claim 127, wherein the polymer is a polyoxyalkylene block copolymer.

168. The method of claim 127, wherein said immunogen composition of the present invention produces at least a humoral immune response.

169. The method of claim 127, wherein said host is human.

170. A vehicle delivery composition for mucosal delivery of a drug, the vehicle delivery composition comprising:
a mist comprising droplets of a flowable medium dispersed in a carrier gas;
the flowable medium comprising an antigen, a biocompatible polymer and a liquid vehicle;
wherein, the polymer interacts with the liquid vehicle to impart reverse thermal viscosity behavior to the composition, so that the viscosity of the composition increases when the temperature of the composition increases over at least some temperature range.

171. The vehicle delivery composition of claim 170, wherein the flowable medium has a reverse-thermal liquid-gel transition temperature that is lower than 40° C.

172. The vehicle delivery composition of claim 171, wherein the flowable medium in the mist is at a temperature of 20° C. or less and the transition temperature is in a range of from 20° C. to 37° C.

173. The vehicle delivery composition of claim 170, wherein the drug comprises an antigen for stimulating a mucosal immune response when the vehicle delivery composition is administered to the host.

174. The vehicle delivery composition of claim 173, wherein the antigen is derived from at least one of bacteria, protozoa, fungus, hookworm, virus and combinations thereof.

175. The delivery vehicle composition of claim 173, wherein the antigen comprises at least one of tetanus toxoid, diphtheria toxoid, a non-pathogenic mutant of tetanus toxoid, a non-pathogenic mutant of diphtheria toxoid and combinations thereof.

176. The delivery vehicle composition of claim 173, wherein the antigen comprises at least one antigen from Bordatella pertussis.

177. The delivery vehicle composition of claim 173, wherein the antigen comprises at least one antigen from influenza virus.

178. The delivery vehicle composition of claim 173, wherein the antigen comprises at least one antigen from M. tuberculosis.

179. The delivery vehicle composition of claim 173, wherein the antigen is derived from at least one causative agent of childhood illness.

180. The delivery vehicle composition of claim 173, wherein the antigen comprises at least one of rotavirus and at least one antigen derived from rotavirus.

181. The delivery vehicle composition of claim 173, wherein the antigen comprises at least one of a polysaccharide, a peptide mimetic of a polysaccharide, or antigen from Neisseria meningitiditis and an antigen from Streptococcus pneumoniae.

182. The delivery vehicle composition of claim 173, wherein the antigen comprises Epstein-Barr virus or at least one antigen derived from Epstein-Barr virus.

183. The delivery vehicle composition of claim 173, wherein the antigen comprises Hepatitis C virus or at least one antigen derived from Hepatitis C virus.

184. The delivery vehicle composition of claim 173, wherein the antigen comprises HIV or at least one antigen derived from HIV

185. The delivery vehicle composition of claim 173, wherein the antigen comprises at least one molecule involved in a mammalian reproductive cycle.

186. The delivery vehicle composition of claim 173, wherein the antigen comprises HCG.

187. The delivery vehicle composition of claim 173, wherein the antigen comprises at least one tumor-specific antigen.

188. The delivery vehicle composition of claim 173, wherein the antigen comprises at least one antigen from a blood-borne pathogen.

189. The delivery vehicle composition of claim 173, wherein the composition contains at least two antigens.

190. The delivery vehicle composition of claim 173, wherein the antigen comprises a first component selected from the group consisting of tetanus toxoid, a nonpathogenic mutant of tetanus toxoid and combinations thereof; and
the antigen comprises a second component selected from the group consisting of diphtheria toxoid, a nonpathogenic mutant of diptheria toxoid and combinations thereof.

191. The delivery vehicle composition of claim 170, wherein the polymer is substantially entirely dissolved in the liquid vehicle.

192. The delivery vehicle composition of claim 191, wherein the drug is substantially entirely dissolved in the liquid vehicle.

193. The delivery vehicle composition of claim 170, wherein the polymer comprises a polyoxyalkylene block copolymer.

194. A method of mucosal delivery of a drug to a host, the method comprising comprising:
introducing a drug delivery vehicle composition into the host, the drug delivery compostion comprising an antigen, a biocompatible polymer and a liquid vehicle, wherein the polymer interacts with the liquid vehicle to impart reverse thermal viscosity behavior to the composition, so that the viscosity of the composition increases when the temperature of the composition increases over at least some temperature range; and
contacting at least a portion of the drug delivery vehicle with a mucosal surface of the host;
wherein during the introducing, the delivery vehicle composition is in the form of disperse droplets in a mist.

195. The method of claim 194, wherein during the introducing, the delivery vehicle composition is in the form of a flowable medium at a first temperature that is lower than the physiologic temperature of the host; and
the delivery vehicle composition converts to a gel form as the delivery vehicle compostion warms inside the host.

196. The method of claim 194, wherein the drug comprises an antigen.

197. The method of claim 195, wherein the polymer comprises a polyoxyalkylene block copolymer.
